(19) United States
(12) Patent Application Publication
(10) Pub. No.: US 2013/0171624 A1
(43) Pub. Date: Jul. 4, 2013

(54) Magnetic Binding Assays Utilizing Time-Resolved Up-Converting Luminescence Detection

(71) Applicant: Aimin He, Alpharetta, GA (US)

(72) Inventor: Aimin He, Alpharetta, GA (US)

(21) Appl. No.: 13/730,814

(22) Filed: Dec. 28, 2012

Related U.S. Application Data
(60) Provisional application No. 61/582,431, filed on Jan. 2, 2012.

(51) Int. Cl. G01N 33/58

(52) U.S. Cl. CPC G01N 33/582
USPC 435/5; 436/501; 435/7.4; 435/7.1

(57) ABSTRACT

This invention describes a general magnetic binding assay method to detect the presence and quantity of analyte in a sample. The method uses magnetic particles for separating and concentrating analytes of interest from complex samples and use time-resolved up-converting fluorescence detection technique to provide highly sensitive detection without using expensive optical components such as band-pass filters. The method uses pulsed long wavelength light for excitation and time-delayed luminescence detection, resulting in little interferences from sample matrices. Furthermore, the usage of long wavelength excitation light requires simpler sample preparation and clean-up such as removal of red blood cells, which otherwise will significantly interfere with excitation efficiency of the fluorescence probes.

This application claims the benefit under 35 USC §119 (e) of U.S. provisional application Ser. No. 61/582,431 filed Jan. 2, 2012, incorporated herein by reference in its entirety.

### BACKGROUND OF THE INVENTION

Magnetic particles have been widely used as a vehicle for analyte separation and concentrating by tagging specific binders on the magnetic particle surfaces to capture the species of interest (e.g., proteins, nucleic acids (DNAs or RNAs), cells, and micro-organisms) from complex samples because they can be easily separated by magnetic fields and require no special and expensive equipment. The captured species can be easily separated from the rest part of the sample through a magnet. The captured species can then be cleaved and released by various means from the magnetic particles. For the purpose of detecting the species of interest, a detection probe tagged with another specific binder may be incubated together with the mixture before magnetic separation so that some of the probes can be captured through the species of interest to form a sandwich complex. The captured probes on the magnetic particles can be then separated from those remaining non-bound detection probes. The captured probes can be measured through various means depending upon the nature of the probes. The amount of the captured probes is proportional to the amount of the species of interest, therefore providing a method for detecting the presence or amount of the species of interest in the sample.

There are several well-known types of binding assays that have been coupled with magnetic particle separation technique to achieve convenient analyte detection. One of the binding assays is immunoassay methods that use immunoreactants labeled with a detectable component so that the analyte can be detected analytically. For “sandwich-type” immunoassays, typically the test sample is first mixed with antibodies that are immobilized on the surface of magnetic particles to capture the analyte of interest. The antibodies are generally specific to the analyte. The magnetic particles are then separated from the rest of the samples followed by mixing with another antibody that are mobile and linked to a label or probe, such as dyed latex, a colloidal metal sol, or a radioisotope, or a fluorescent dye, or an enzyme. The antibody is also specific to the analyte on a different epitope. The magnetic particles were then again separated from the non-bound label or probe through a magnet. The signal of the label that is captured together with the magnetic particles is then measured and correlated with a standard curve to obtain the amount of the analyte. In this manner, magnetic immunoassays can provide a fast and simple technique to determine the presence or absence of the species. In such assays, various signal-generating mechanisms have been used, including color (absorption and reflectance), fluorescence, chemilluminescence, radioactivity and enzymes.

Similarly, nucleic acids (DNA and RNA) hybridization assay can also be coupled with the magnetic separation to achieve detection and quantitifcation of DNA and RNA in a sample. Various signal-generating mechanisms can be used, including color (absorption and reflectance), fluorescence, chemilluminescence, radioactivity and enzymes for magnetic DNA/RNA hybridization assays.

However, all of those current signal generating mechanisms have significant limitations for magnetic binding assays. For instances, absorbance based color detection has low sensitivity. The dark and brown color of most commercial magnetic particles interferes with the absorbance or reflectance measurements of the colored probes. Conventional fluorescence measurement can be interfered by autofluorescence from most of complex biological samples and required very expensive instruments. Furthermore, the magnetic particles often have significant absorption of UV or visible excitation light where most of fluorescence probes are excited. The time-resolved fluorescence can eliminate the interference of background fluorescence and autofluorescence, and can be cheaper. However, all the useful probes suitable for time-resolved luminescence measurement need to be excited close to UV or shorter than 450 nm, where most of biological samples have significant absorption. The absorption of the excitation light by analyte itself and sample matrices in close to UV region significantly compromise the accuracy of the results. The short wavelength excitation light may also damage the analytes through photo-induced oxidation. Accordingly, there are still needs for building an improved method for measurements of fluorescence. A need currently exists for a binding assay that uses a signal detection technique that is cheap, accurate and sensitive.

### SUMMARY OF THE INVENTIONS

In accordance with one embodiment of the present invention, an assay method for detecting the presence or quantity of an analyte in a sample is disclosed. The assay method comprises of (a) contacting a sample containing an analyte of interest with magnetic particles conjugated with a first specific binding member and a detection probe tagged with a second specific binding member. The detection probe is an up-converting luminescent label that is capable of emitting strong luminescence of more than 5 μs lifetime at a shorter wavelength than the wavelength of an excitation illumination. The first specific binding member and second specific binding members bind specifically with different epitopes of the analyte to form a sandwich complex; (b) separate all the magnetic particles from the rest of the sample using a magnet device, where the magnetic particles include the sandwich complexes; (c) exciting the detection probes in the sandwich complexes using pulsed illuminations at a first wavelength to obtain a detection signal by collecting and measuring the luminescence at a second wavelength after a certain period of time has elapsed following each illumination pulse. The second wavelength is shorter than the first wavelength; (d) sum up all the measured signals from all the excitation pulses to obtain a detection signal and compare the detection signal with a calibration curve to obtain the amount of analyte in the sample. The detection signal is proportional to the detection signal.

In accordance with another embodiment of the present invention, an assay method for detecting the presence or quantity of an analyte in a sample is disclosed. The assay method comprises of (a) contacting a sample containing an analyte of interest with magnetic particles conjugated with a first specific binding member on their surfaces and a known amount of detection probe tagged with the analyte or an analyte analog. The first binding member binds specifically with the analyte and analyte analog. The detection probe is capable of emitting luminescence of more than 5 μs lifetime at a shorter wavelength than the wavelength of an excitation illumination. The detection probe tagged with the analyte or analyte analog competes with the analyte in the sample for a limited amount of the first binding members conjugated to the magnetic particles to form a complex; (b) separate the magnetic particles from the rest of the sample using a magnet device, where the magnetic particles include the complex between the magnetic particle tagged with the first binding member and the detection probe tagged with the analyte or analyte analog; (c) exciting the detection probes in the complexes using pulsed illuminations at a first wavelength to obtain a detection signal by collecting and measuring the luminescence at a second wavelength after a certain period of time has elapsed following each illumination pulse. The second wavelength is shorter than the first wavelength; (d) sum up all the measured signals from all the excitation pulses to obtain a detection signal and compare the detection signal with a calibration curve to obtain the amount of analyte in the sample. The detection signal is inversely proportional to the detection signal.

Other features and aspects of the present invention are disclosed in greater detail below.

### BRIEF DESCRIPTION OF THE DRAWINGS

FIG. 1 is a schematic diagram of one embodiment of a sandwich complex binding assay of the present invention;

FIG. 2 is a schematic diagram of signal detection modes.

FIG. 3 is a schematic diagram of one embodiment of a competitive binding assay of the present invention

FIG. 4: is a schematic diagram of one embodiment of a competitive binding assay of the present invention

FIG. 5: Scanning electron micrograph of the up-converting luminescence particles obtained in Example 1.

FIG. 6 is a graph of time-resolved up-converting excitation and emission spectra of the conjugated luminescence probes obtained in Example 2.

FIG. 7 is the up-converting emission decay for the conjugated probes obtained in Example 2.

### DETAILED DESCRIPTION OF REPRESENTATIVE EMBODIMENTS

The term “analyte” generally refers to a substance to be detected. For instance, analytes can include antigens, haptens, antibodies, and combinations thereof. Analytes include, but are not limited to, toxins, organic compounds, proteins, peptides, microorganisms, amino acids, nucleic acids, hormones, steroids, vitamins, drugs, bacteria, virus particles and metabolites of or antibodies to any of the above substances.

The term “sample” generally refers to a material suspected of containing the analyte. The sample can be used directly as obtained from the source or following a pretreatment to modify the character of the sample. The test sample can be derived from any biological source, such as a physiological fluid, including, blood, saliva, ocular lens fluid, cerebral spinal fluid, sweat, urine, milk, ascites fluid, raucous, synovial fluid, peritoneal fluid, amniotic fluid or the like. The test sample can be pretreated prior to use, such as preparing plasma from blood, diluting viscous fluids, and the like. Besides physiological fluids, other liquid samples can be used such as water, food products and the like for the performance of environmental or food production assays.

### DETAILED DESCRIPTION

In general, the present invention is partly based on the discovery that the up-converting fluorescence can overcome some of the autofluorescence and background light scattering issues encountered by the fluorescence technique, but not completely eliminate them. Furthermore, up-converting fluorescence measurement still requires expensive optical filter to separate the excitation lights and fluorescence signals. Because of needs for optical components for fluorescence separation, it is a challenge to build a compact, low-cost portable apparatus for measurements of up-converting fluorescence.

In general, the present invention is directed to a magnetic binding assay (e.g., sandwich assay, competitive assay, etc) for detecting the presence and quantity of an analyte in a sample. The magnetic binding assay uses up-converting luminescence probes that are capable of generating a luminescence signal of a long luminescence lifetime at a shorter wavelength when the probes are excited by pulsed illuminations of a longer wavelength. The up-converting luminescence was collected and measured at a certain period of time after the excitation by pulsed illuminations. The amount of the analyte in the sample is proportional (directly or inversely) to the time-resolved up-converting luminescence.

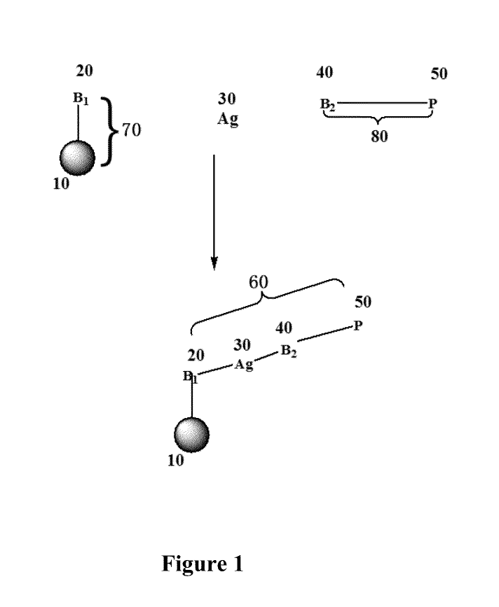

One embodiment of the present invention will be described in more detail below. Referring to FIG. 1, the present assay includes a magnetic particle 10 that is tagged with a first specific binding member 20 to form a magnetic particle conjugate 70. The present invention further includes a probe conjugate 80 that comprises of a second specific binding member 40 that is tagged with a detection probe 50. The detection probe 50 is referred to an up-converting luminescence label that can generate up-converting luminescence of a long luminescence lifetime at a shorter wavelength when the probe 50 absorbs two photons of a longer wavelength through excitation by pulsed illuminations. The first specific binding member 20 and the second specific binding member 40 bind specifically with different epitopes of an analyte of interest to form a sandwich complex 60.

To carry out the assay, a sample containing the analyte is first in contact with the magnetic particle conjugate 70 and the probe conjugate 80. The analyte Ag 30 binds with the first binding member 20 of the magnetic particle conjugate 70 and the second binding member of the probe conjugate 80 to form a sandwich complex 60. The complex 60 is then separated through a magnet device from the remaining portion of the sample that are not magnetic-responsive, such as those probe conjugates 80 that are not complexed to the magnetic conjugate 60 through the analyte Ag 30.

In general, the contact can be carried out in two different ways. One way is to mix both magnetic particle conjugates 70 and the probe conjugates 80 together with the sample. Another way is to mix the magnetic particle conjugate 70 first with the sample containing the analyte Ag 30, and then separate the magnetic particles in the sample by a magnet device. The separated magnetic particle conjugates 70, either free magnetic conjugates 70 or those complexed with the analyte Ag 30, are then mixed with the probe conjugates 80 to form the complex 60. The magnetic particles are then separated by a magnetic device. Regardless of the mixing methods, the time-resolved up-converting luminescence of the probe 50 in the complex 60 can be directly measured on the capturing magnet device to obtain a detection signal by an apparatus 90. Alternatively, the captured complex 60 can be first re-suspended in a solution followed by measuring the time-resolved up-converting luminescence of the captured probe 50 by the apparatus 90. The measured time-resolved up-converting luminescence detection signal is compared with a calibration curve to obtain the quantity of the analyte in the sample. The calibration curve is in general created by plotting the time-resolved up-converting luminescence detection signals versus the analyte concentration for a range of known analyte concentrations. To determine the quantity of analyte in an unknown test sample, the detection signal is then converted to analyte concentration according to the calibration curve.

Generally, the magnetic particle 10 is made of material that is “magnetically responsive”. The particle is attracted or repulsed or has a detectable magnetic susceptibility or induction. For instance, some examples of suitable magnetically responsive materials that can be used to impart magnetic properties to a probe include, but are not limited to, paramagnetic materials, superparamagnetic materials, ferromagnetic materials, ferrimagnetic materials, and metamagnetic materials. Specific examples are metals such as iron, nickel, cobalt, chromium, manganese, and the like; lanthanide elements such as neodymium, erbium, and the like; alloys such as magnetic alloys of aluminum, nickel, cobalt, copper and the like; oxides such as ferric oxide (Fe₃O₄), ferrous oxide (Fe₂O₃), chromium oxide (CrO₂), cobalt oxide (CoO), nickel oxide (NiO₂), manganese oxide (Mn₂ O₃) and the like; composite materials such as ferrites and the like; and solid solutions such as magnetite with ferric oxide and the like. The mean diameter of the particulate probes may generally vary as desired depending on factors such as the type of particle chosen, the pore size of the membrane, and the membrane composition. For example, in some embodiments, the mean diameter of the particulate probes can range from about 0.01 microns to about 1,000 microns, in some embodiments from about 0.01 microns to about 100 microns, and in some embodiments, from about 0.01 microns to about 10 microns. In one particular embodiment, the particulate probes have a mean diameter of from about 1 to about 2 microns. Generally, the particles are substantially spherical in shape, although other shapes including, but not limited to, plates, rods, bars, irregular shapes, etc., are suitable for use in the present invention. As will be appreciated by those skilled in the art, the composition, shape, size, and/or density of the particles may widely vary.

The first specific binding member 20 and the second specific binding member 40 generally refer to a member of a specific binding pair, i.e., two different molecules where one of the molecules chemically and/or physically binds to the second molecule. For instance, immunoreactive specific binding members can include antigens, haptens, aptamers, antibodies, and complexes thereof, including those formed by recombinant DNA methods or peptide synthesis. An antibody can be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and the like. Furthermore, specific binding pairs can include members that are analogs of the original specific binding member. For example, a derivative or fragment of the analyte, i.e., an analyte-analog, can be used so long as it has at least one epitope in common with the analyte.

The specific binding members 20 and 40 can generally be attached to the magnetic particle 10 and the probe 50, respectively, using a variety of well-known techniques. For instance, covalent attachment of the specific binding members 20 to the magnetic particle 10 and the specific binding member 40 to the probe 50 can be accomplished using carboxylic, amino, aldehyde, bromoacetyl, iodoacetyl, thiol, epoxy and other reactive or linking functional groups, as well as residual free radicals and radical cations, through which a protein coupling reaction can be accomplished. A surface functional group can also be incorporated as a functionalized co-monomer because the surface of the microparticle can contain a relatively high surface concentration of polar groups. In addition, although microparticle probes are often functionalized after synthesis, the microparticles are capable of direct covalent linking with a protein without the need for further modification. For example, covalent attachment of the first binding member antibody to a carboxylic acid functionalized magnetic particle can be accomplished by two steps. The first step of conjugation is activation of carboxylic groups on the probe surface using carbodiimide. In the second step, the activated carboxylic acid groups are reacted with an amino group of an antibody to form an amide bond. Besides covalent bonding, other attachment techniques, such as adsorption, may also be utilized in the present invention.

The detection probe 50 is referred to an up-converting luminescence label that can generate luminescence at a shorter wavelength of a long luminescence lifetime when the probe 50 is excited by an illumination of a longer wavelength to simultaneously absorb two photons. The luminescence lifetime of the up-converting luminescence probes is generally longer than 5 μs. More specifically the luminescence lifetime of the probe 50 ranges from 20 μs to 3000 μs. The detection probes 50 are configured to allow time-resolved up-converting luminescence detection. Time-resolved up-converting luminescence involves exciting the probe 50 with a short pulse of light at a longer wavelength, typically far red or near IR, to allow two-photon absorption, then typically waiting a certain time (e.g., between approximately 20 to 200 microseconds) after excitation before measuring the remaining long-lived luminescence signal at a shorter wavelength. By exciting the probe at far red or near IR, absorption of the excitation photons by samples including analytes and matrices, and autofluorescence of sample matrices can be significantly minimized. As results, the complex samples don't need to be processed or pre-cleaned and can be directly measured in some cases. Furthermore, time-resolved up-converting luminescence measurement can eliminate any short-lived fluorescent background signals and scattered excitation radiation to result in sensitivities that are 2 to 4 orders greater than conventional luminescence detection techniques. In addition to higher detection sensitivity and no need to pre-clean the complex samples, the time-resolved up-converting luminescence detection apparatus does not need expensive optical components for luminescence signal separation of the probe from the background. Therefore low cost detection apparatus is possible.

The desired probe for time-resolved up-converting luminescence should have good quantum efficiency of up-converting luminescence with a relatively long emission lifetime. Namely the probe can desirably have strong two-photon absorption of far red or near IR light (longer wavelength) and emit luminescence at a visible light region (shorter wavelength). Therefore, the luminescence has an anti-Stoke shift. The long luminescence lifetime is also important and this is desired so that the probe emits its signal well after any short-lived background signals dissipate. Furthermore, a long fluorescence lifetime makes it possible to use low-cost circuitry for time-gated fluorescence measurements. For example, the probe used in the present invention may have a luminescence lifetime of greater than about 5 microsecond, in some embodiments greater than about 10 microseconds, in some embodiments greater than about 50 microseconds, and in some embodiments, from about 100 microseconds to about 1000 microseconds. The term “anti-Stokes shift” is generally defined as the displacement of spectral lines or bands of luminescent radiation to a shorter emission wavelength than the excitation lines or bands.

One class of suitable probes for up-converting luminescent magnetic binding assays is lanthanide chelates of samarium (Sm (III)), dysprosium (Dy (III)), europium (Eu (III)), and terbium (Tb (III)). Such chelates can absorb two photons of far red or near IR simultaneously and exhibit strongly blue-shifted, narrow-band, long-lived emission after excitation of the chelate at substantially longer wavelengths. For example, the up-converting luminescence of europium chelates is long-lived, with lifetimes of about 20 to about 1000 microseconds, as compared to about 1 to about 100 nanoseconds for typical fluorescent labels. One suitable europium chelate is N-(p-isothiocyanatobenzyl)-diethylene triamine tetraacetic acid-Eu⁺³.

In additional to up-converting luminescence molecules as probes, the probes can also be in a variety of forms. For example, the probes may be in a form of polymers, liposomes, dendrimers, and other micro- or nano-scale structures that are tagged or encapsulated with up-converting luminescent molecules. In addition, the probes may be in a form of microparticles or microbeads. The up-converting luminescent molecules are referred as molecules such as lanthanide chelates that are capable of generating strong up-converting luminescence of relatively long lifetime with blue-shift relative to excitation illumination upon excitation. For example, in one embodiment, latex microparticles that are encapsulated with up-converting luminescent molecules are utilized. The latex microparticles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and the like, or an aldehyde, carboxyl, amino, hydroxyl, or hydrazide derivative thereof.

When particles are utilized as probes, the mean diameter of the particles may generally vary as desired. For example, in some embodiments, the mean diameter of the particulate labels can range from about 0.01 microns to about 1,000 microns, in some embodiments from about 0.01 microns to about 100 microns, and in some embodiments, from about 0.01 microns to about 10 microns. In one particular embodiment, the particles have a mean diameter of from about 0.1 to about 2 microns. Generally, the particles are substantially spherical in shape, although other shapes including, but not limited to, plates, rods, bars, irregular shapes, etc., are suitable for use in the present invention. As will be appreciated by those skilled in the art, the composition, shape, size, and/or density of the particles may widely vary.

Another class of suitable probes for the present invention is phosphor particles with a crystalline matrix doped with lanthanide ions. Examples of the lanthanide ion doped phosphor particles include Yb/Er or Yb/Tm co-doped NaYF4 nanoparticles that have efficient infrared-to-visible up-converting luminescence. The up-converting luminescence of those particles have relatively long lifetime that is suitable for time-resolved up-converting luminescence measurements. In addition to those lanthanide ion doped phosphor particles, latex particles that are encapsulated with the lanthaonide-doped phosphor nanocrystals are also useful probes for the present invention.

The time-resolved up-converting luminescence detection is designed to reduce background signals from the emission source or from scattering processes (resulting from scattering of the excitation radiation) by taking advantage of the long lived luminescence characteristics of up-converting luminescence probes such as lanthanide chelates of europium (Eu (III)) and terbium (Tb (III)), and particles encapsulated with those chelates and lanthanide ion-doped phosphor crystals. The time-resolved up-converting luminescence detection which typically uses long wavelength lights or photons for tow-photon excitation is further designed to avoid use of short wavelength excitation of conventional time-resolved fluorescence detection techniques which is often harmful to cells and other biological species. The short wavelength excitation used for conventional time-resolved fluorescence detection technique has limited penetration depth through most of biological matrices and other types of materials, resulting in non-optimal excitation efficiency. The up-converting luminescence probes can exhibit strongly blue-shifted, narrow-band, long-lived emission after excitation of the probe at substantially longer wavelengths. The use of pulsed excitation at far red and near IR region and time-gated detection allows for specific detection of the luminescence from the probe only, rejecting emission from other species present in the sample that are typically shorter-lived. Use of long-wavelength pulsed excitation light (>650 nm) can avoid damage of biological samples such as cells, and interference of probe excitation from sample absorption of excitation photons. Use of long-wavelength pulsed excitation light (>650 nm) can improve the penetration depth of the excitation light and increase the excitation effectiveness. Therefore, the time-resolved up-converting luminescence detection technique of the present invention has multiple advantages over conventional time-resolved fluorescence detection technique and conventional up-converting fluorescence detection technique.

One embodiment of the apparatus 90 for measuring time-resolved luminescence includes an excitation source and a photodetector. The excitation source provides pulsed illuminations at far red or near IR region to excite the detection probes so that probes can simultaneously absorb two-photons effectively. Various excitation sources may be used in the present invention, including light emitting diodes (LED), flashlamps, as well as other suitable sources. Excitation illumination may also be multiplexed and/or collimated; for example, beams of various discrete frequencies from multiple coherent sources (e.g., lasers) can be collimated and multiplexed using an array of dichroic mirrors. Further, illuminations are pulsed, or may combine continuous wave (CW) and pulsed illuminations where multiple illumination beams are multiplexed (e.g., a pulsed beam is multiplexed with a CW beam), permitting signal discrimination between luminescence induced by the CW source and luminescence induced by the pulsed source.

The examples of suitable detectors that can be used in the present invention include, but not limited to, photomultiplier devices; photodiodes, such as avalanche photodiodes, silicon photodiodes, etc.; high speed, linear charge-coupled devices (CCD), CID devices, or CMOS based imagers; and the like. In one embodiment, the apparatus 90 utilizes a silicon photodiode for luminescence detection. Silicon photodiodes are advantageous in that they are inexpensive, sensitive, capable of high-speed operation (short risetime/high bandwidth), and easily integrated into most other semiconductor technology and monolithic circuitry. In addition, silicon photodiodes are physically small, which enables them to be readily incorporated into a portable system. If silicon photodiodes are used, then the wavelength range of the luminescence emission should be within their range of sensitivity, which is 400 to 1100 nanometers. Another detector option is a CdS (cadmium sulfide) photoconductive cell, which has the advantage of having a spectral sensitivity similar to that of human vision (photopic curve) that may make rejection of the reflected excitation radiation easier.

The apparatus 90 includes various timing circuitry used to control the pulsed excitation of the excitation source and the measurement of the emitted luminescence. For instance, a clock source (e.g., a crystal oscillator) can be employed to provide a controlled frequency source to other electronic components in the apparatus 90. In this particular embodiment, for instance, the oscillator may generate a 20 MHz signal, which is provided to an LED driver/pulse generator and to an ND converter. The clock signal from oscillator to A/D converter controls the operating speed of A/D converter. It should be appreciated that a frequency divider may be utilized in such respective signal paths if the operating frequency of ND converter or if the desired frequency of the clock input to LED driver/pulse generator is different than 20 MHz. Thus, it should be appreciated that the signal from oscillator may be modified appropriately to provide signals of a desired frequency. In some embodiments, a signal from oscillator may also be provided to microprocessor to control its operating speed. Additional frequency dividers may be utilized in other signal paths in accordance with the present invention.

The apparatus 90 also include a microprocessor to provides control input to pulse generator such that the 20 MHz signal from oscillator is adjusted to provide a desired pulse duration and repetition rate (for example, a 1 kHz source with a 50% duty cycle). The signal from pulse generator may then be provided to the excitation source, controlling its pulse repetition rate and duty cycle of illumination. In some embodiments, a transistor may be provided in the signal path to excitation source, thus providing a switching means for effecting a pulsed light signal at excitation source.

As described above, the pulsed light excites the up-converting luminescence probes. After the desired response time (e.g., about 20 to about 200 microseconds), the detector detects the luminescence signal emitted by the excited probes and generates an electric current representative thereof. This electric current may then be converted to a voltage level by a high-speed transimpedance preamplifier, which may be characterized by a relatively low settling time and fast recovery from saturation. The output of the preamplifier may then be provided to the data input of A/D converter. Additional amplifier elements (such as a programmable gain amplifier) may be employed in the signal path after preamplifier and before A/D converter to yield a signal within an appropriate voltage range at the trailing edge of the excitation pulse for provision to the A/D converter. A/D converter may be a high-speed converter that has a sample rate sufficient to acquire many points within the fluorescence lifetime of the subject fluorescent labels. The gain of the preamplifier may be set such that data values drop below the maximum A/D count (e.g., 2047 for a 12-bit converter) on the trailing edge of the excitation pulse. Data within the dynamic range of A/D converter would then be primarily representative of the desired fluorescence signal. If the sample interval is short compared with the rise-time and fall-time of the excitation pulse, then the gain of preamplifier may be set to ensure that signal values within the upper ½ or ¾ of the dynamic range of A/D converter correspond to the trailing edge of the emission pulse.

A/D converter samples the signal from preamplifier and provides it to the microprocessor where software instruction is configured for various processing of the digital signal. An output from the microprocessor is provided to the A/D converter to further control when the detected fluorescence signal is sampled. Control signals to preamplifier and to A/D converter may be continuously modified to achieve the most appropriate gain, sampling interval, and trigger offset. It should be appreciated that although the A/D converter and the microprocessor are depicted as distinct components, commercially available chips that include both such components in a single module may also be utilized in the present invention. After processing, the microprocessor may provide at least one output indicative of the fluorescence levels detected by the detector. One such exemplary output is provided to a display, thus providing a user with a visual indication of the fluorescence signal generated by the label. Display may provide additional interactive features, such as a control interface to which a user may provide programmable input to microprocessor.

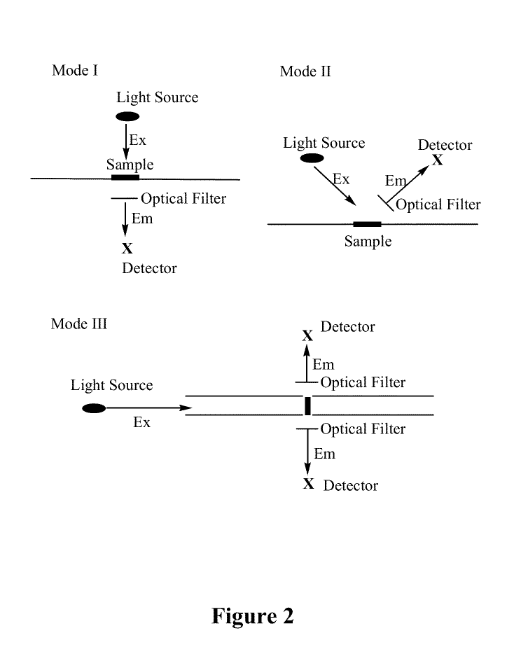

The detection mode of the separated complex 60 can be varied as described in FIG. 2. In the detection mode I, the pulsed excitation illumination is directly shined on the sample and the photodetector is placed on the opposite side of the illumination source. In Mode II, the pulsed excitation illumination is shined on the sample in a certain angle and the photodetector is placed on the same side of the illumination source orientated at a certain angle to collect and measure the up-converting luminescence from the probe in a time-delayed manner. In Mode III, the pulsed excitation illumination is directly shined in a 90° angle on the sample and the photodetector is placed perpendicular to the illumination direction.

Regardless of the detection mode, it is well known in the art that at least one optical filter is needed to separate the excitation illumination from the up-converting luminescence for conventional unconverting luminescence measurements for all the three modes. Conventional unconverting luminescence measurements are considered to be not practical for Mode I because the excitation illumination is normally magnitudes more intense than the up-converting luminescence and optical filters are difficult to completely eliminate all the excitation illumination directly shined on the photodetector. As a result, the detection background is significant and detection sensitivity is limited. However, Mode I is practical for time-resolved up-converting luminescence detection technique because the separation of the up-converting luminescence signal from the illumination is achieved through a time delay. Therefore, the excitation illumination will have a minimal interference on the luminescence measurement if the excitation illumination has delayed to a background level during the luminescence measurement.

The inventor's investigation has discovered that magnetic binding assays using time-resolved up-converting luminescence detection techniques have advantages over the conventional time-resolved luminescence detection techniques, although both techniques use pulsed excitation illuminations and time-delayed measurements to separate the background from the luminescence signals. It is known in the art that the existing detection probes suitable for conventional time-resolved luminescence detection techniques are limited to the lanthanide chelates, platinum and palladium chelates, and particles encapsulated with those chelates. Although those probes have strong luminescence and long luminescence lifetime that are very important for any time-resolved luminescence detection techniques, all those probes can be effectively excited only by illuminations of less than 450 nm. This short wavelength excitation at less than 450 nm for conventional time-resolved luminescence detection technique has significant limitations. For instances, the strong illuminations of less than 450 nm is generally more expensive and is more harmful than their longer wavelength counterparts to analytes such as proteins and nucleic acids. Many samples and sample matrices have significant absorption in this wavelength region, therefore, interfering with the efficient absorption and excitation for the detection probes. In many cases, the analytes may also have significant absorption in this region such as proteins and nucleic acids. The exposure of those samples and analytes to the strong excitation illuminations may subject those analytes and samples to degradation and results in inaccurate measurements. This is a very significant issue for magnetic binding assays using conventional time-resolved luminescent detection techniques because most of commercially available magnetic particles have strong absorption at less than 450 nm, therefore, also interfering with the consistent and effective excitation of the probes. The inventor of this invention has found that those issues discussed above for conventional time-resolved luminescence detection techniques do not exist for time-resolved up-converting luminescence detection techniques for magnetic binding assays, because the probes are effectively excited by either far visible light or near IR illuminations. Those far red or near IR illuminations are safe to most of analytes and are often cheaper than their short wavelength counterparts. Most of samples and matrices does not have strong absorption and therefore have minimal interference with the effective excitation of the probes. The magnetic particles have much weaker absorption in the far red and near IR region, therefore also presenting minimal problems for effectiveness in probe excitation.

In general, up-converting excitation (two photo excitation) efficiency is very low, even for those probes considered to be the best in the class. Therefore, time-resolved up-converting luminescence detection techniques are not considered to be viable detection techniques in comparison with conventional up-converting luminescence detection techniques because the time-resolved up-converting luminescences uses pulsed excitation illumination rather than continuous illuminations, which makes the overall signal too weak to be useful. However, the inventor has recently developed highly bright up-converting luminescence probes that make the detection technique viable.

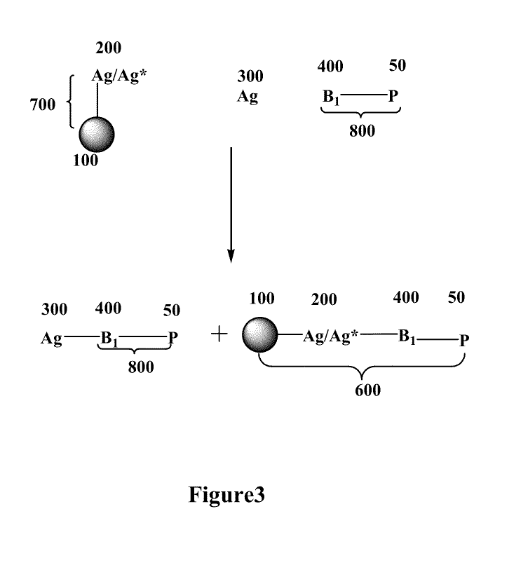

Another embodiment of the present invention is a magnetic competitive binding assay using time-resolved up-converting luminescence detection technique. Referring to FIG. 3, the present assay includes a magnetic particle 100 that is tagged to a first specific binding member 200 to form a magnetic particle conjugate 700. The first specific binding member is either an analyte or analyte analog. The present invention further comprises of a probe conjugate 800. The probe conjugate 800 comprises of a second specific binding member 400 that is tagged with a detection probe 50, either covalently or physically. The second binding member is specific for the analyte and analyte analog. The detection probe 50 is referred to an up-converting luminescence label that can generate luminescence of a long luminescence lifetime at a shorter wavelength when the probe 50 is excited by an illumination of a longer wavelength to absorb two photons. The up-converting luminescence lifetime of the probes is generally longer than 5 microseconds. More specifically the luminescence lifetime of the probe 50 ranges from 20 microseconds to 3000 microseconds. Examples of the up-converting luminescence probes include, but not limited to, the molecules of lanthanide chelates, latex particles that are encapsulated with the lanthanide chalates, lanthanide ion-doped phosphor nanocrystals, latex particles encapsulated with the phosphor nanocrystals.

Further referring to FIG. 3 to carry out the assay, a sample is first in contact with the probe conjugate and the magnetic particle conjugate, either stepwise through sequentially mixing and separation, or simultaneously mixing together. Regardless of the mixing manners, if an analyte, Ag 300, is present, the analyte competes with the magnetic particle conjugates for a limited amount of the probe conjugates. As a result, the amount of the probe conjugate that are bound with the magnetic particle conjugates decreases in comparison with the situation where the sample has no analyte. The bounded probe conjugate to the magnetic particle conjugates are then separated from the non-bound probe conjugates by a magnetic device. The separated probe conjugates are then measured by time-resolved up-converting luminescence detection technique through an apparatus. In general, the mixing can be carried out in two different ways. One way is to mix magnetic particle conjugates with the sample first, followed by mixing with the probe conjugates followed by separation through a magnetic device. Another way is to mix the magnetic particle conjugate and probe conjugates together with the sample followed by magnetic separation.

Regardless of the mixing and separation methods, the time-resolved up-converting luminescence of the complex 600 can be directly measured on the capturing magnetic device for the signals of the captured probes by an apparatus. Alternatively, the captured complex 600 can be first re-suspended in a solution followed by measuring the time-resolved up-converting luminescence of the captured probes.

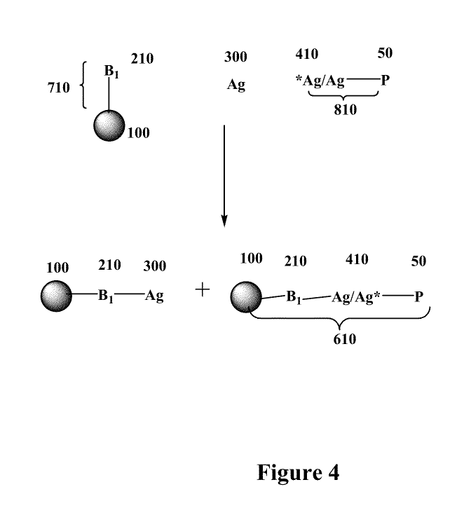

Another embodiment of magnetic competitive binding assay using time-resolved up-converting luminescence detection technique is described in FIG. 4. The present assay includes a magnetic particle 100 that is tagged to a first specific binding member 210 to form a magnetic particle conjugate 710. The first specific binding member is a specific binding member for an analyte. The present invention further comprises of a probe conjugate 810. The probe conjugate 810 comprises a second specific binding member 410 that is tagged with a detection probe 50, either covalently or physically. The second binding member is either analyte and analyte analog. The detection probe 50 is referred to an up-converting luminescence label that can generate luminescence at a shorter wavelength of a long luminescence lifetime when the probe 50 is excited by an illumination of a longer wavelength to absorb two photons. The luminescence lifetime of the up-converting luminescence probes is generally longer than 5 microseconds. More specifically the luminescence lifetime of the probe 50 ranges from 20 microseconds to 3000 microseconds. Examples of the up-converting luminescence probes include, but not limited to, the molecules of lanthanide chelates, latex particles that are encapsulated with the lanthanide chalates, lanthanide-doped phosphor nanocrystals, latex particles encapsulated with the phosphor nanocrystals.

Further referring to FIG. 4 to carry out the assay, a sample is first in contact with the probe conjugate and the magnetic particle conjugate, either stepwise through sequentially mixing and separation, or simultaneously mixing together. Regardless of the mixing manners, if an analyte, Ag 300, is present, the analyte competes with the probe conjugates for a limited amount of the magnetic particle conjugates. As a result, the amount of the probe conjugate that are bound with the magnetic particle conjugates decreases in comparison with the situation where he sample has no analyte. The bounded probe conjugate to the magnetic particle conjugates are then separated from the non-bound probe conjugates by a magnetic device. The separated probe conjugates are then measured by time-resolved up-converting luminescence detection technique through an apparatus. In general, the mixing can be carried out in a two different ways. One way is to mix magnetic particle conjugates with the sample first, followed by mixing with the probe conjugates followed by separation through a magnetic device. Another way is to mix the magnetic particle conjugate and probe conjugates together with the sample followed by magnetic separation.

Regardless of the mixing and separation methods, the time-resolved up-converting luminescence of the complex 610 can be directly measured on the capturing magnetic device for the signals of the captured probes by an apparatus. Alternatively, the captured complex 610 can be first re-suspended in a solution followed by measuring the time-resolved up-converting luminescence of the captured probes. The measured time-resolved up-converting luminescence detection signal is compared with a calibration curve to obtain the quantity of the analyte in the sample. The calibration curve is in general created by plotting the luminescence detection signals versus the analyte concentration for a range of known analyte concentrations. To determine the quantity of analyte in an unknown test sample, the signal is then converted to analyte concentration according to the calibration curve.

The present invention may be better understood with reference to the following examples.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

### Example 1

### Preparation of Up-Converting Luminescence Probes

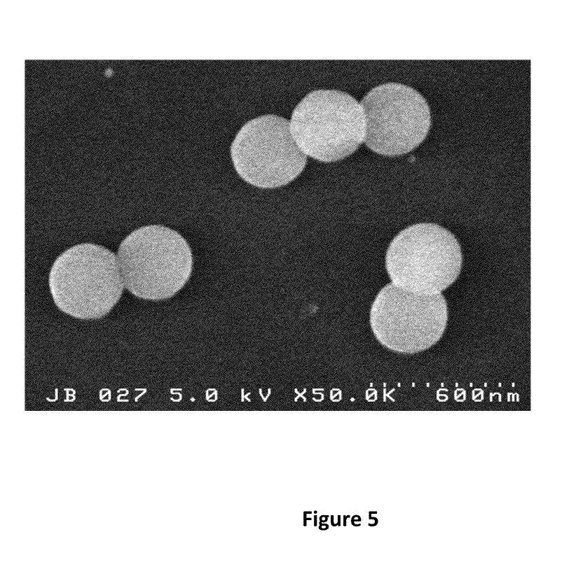

50 mg of carboxylic acid-functionalized latex particles (0.33 mm in diameter from Bangs Laboratories) in 500 μl aqueous solution is added with 2 ml ethanol to under stirring. The particle suspension is slowly added with an appropriate amount (e.g., 1% weight of the latex particles) of a proprietary europium chelate in ethylene chloride (e.g., 3% weight of the total solvents) under stirring. The mixture is stirred for half hour. Then a proper amount of water (e.g., four times amount of the total initial solvents) is slowly added to the stirring mixture over a certain period of time (e.g., 2 hours). After completing the addition of water, most of the ethanol in the mixture is removed through a rotavapor. The particles are then washed twice by 90% ethanol through centrifugation. The particles are then washed twice with water. The washed particles are then suspended by sonication in tris buffer containing 0.5% Tween 20 to make 5% suspension. FIG. 5 shows the scanning electron micrograph of the probe particles.

### Example 2

### Conjugation of Antibody to the Up-Converting Luminescence Probes to Make Probe Conjugates

200 μl of the probes prepared in example 1 is washed once by 1.5 ml carbonate buffer and twice by Mes buffer (PH=4.3) through centrifugation. The washed particles are re-suspended in 0.1 ml Mes buffer and 6.2 mg carbodiimide (from Polysciences, Inc.) dissolved in 0.1 ml Mes buffer is added to the suspended particles. The mixture is allowed to react at room temperature for 30 minutes on a shaker. The activated particles are then washed twice by borate buffer. The activated particles are re-suspended in 0.185 ml borate buffer and 15 μl of LH a monoclonal antibody (LH a Mab, 9.8 mg/ml from Fitzgerald Industrial International, Inc.) is added. The reaction mixture is allowed to react on a shaker overnight. The particles are then collected and incubated in 0.2 ml of 0.1 M ethanolamine under gentle shaking for 15 minutes.

The particles are then washed twice by PBS and are stored at 4° C. in storage buffer. The storage buffer contains 0.1 M PBS, 0.15 M NaCl, 1% BSA, and 0.1% NaN₃. The probe conjugates are designated as α-Mab-P.

### Example 3

### Conjugation of Antibody to Magnetic Particles to Make Magnetic Particle Conjugates

100 μl of 10% carboxylated magnetic particles (1.5 μm, from Bangs Laboratories) is washed once by 1.5 ml carbonate buffer and twice by Mes buffer (PH=4.3) through a magnetic separator. The washed particles are re-suspended in 0.1 ml Mes buffer and 6.2 mg carbodiimide (from Polysciences, Inc.) dissolved in 0.1 ml Mes buffer is added to the suspended particles. The mixture is allowed to react at room temperature for 30 minutes on a shaker. The activated particles are then washed twice by borate buffer. The activated particles are re-suspended in 0.185 ml borate buffer and 15 μl of LH β monoclonal antibody (LH β Mab, Fitzgerald Industrial International, Inc.) is added. The reaction mixture is allowed to react on a shaker overnight. The particles are then collected and incubated in 0.2 ml of 0.1 M ethanolamine under gentle shaking for 15 minutes. The particles are then washed twice by PBS and are stored at 4° C. in storage buffer. The storage buffer contains 0.1 M PBS, 0.15 M NaCl and 1% BSA. The probe conjugates are designated as MP-β-Mab (10 mg/ml).

### Example 4

### Time-Resolved Up-Converting Excitation and Emission Spectra of Probe Conjugates

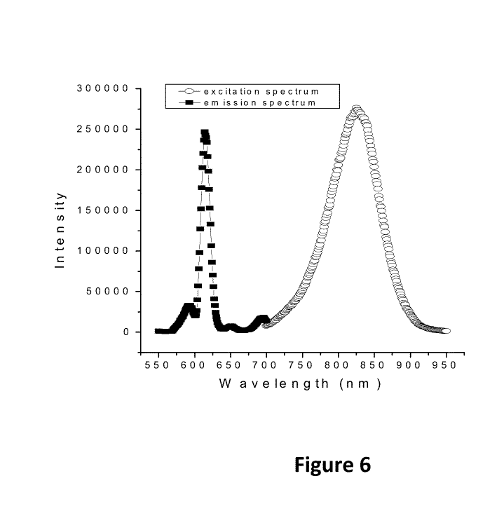

10 ng of the probe conjugates prepared in Example 2 is suspended in 600 μl water to in a cell. The time-resolved up-converting excitation and fluorescence spectra are measured on a fluorometer equipped with a time-resolved capability. The spectra are shown in FIG. 6 by using the following measuring parameters. For time-resolved up-converting fluorescence spectrum: excitation at 870 nm, sample window at 50 μs, time-per-flash at 100 μs, initial delay at 0.01 μs, number of flash at 10, and number of scan at 10, fluorescence collection from 500 nm to 800 nm. For time-resolved up-converting excitation spectrum: emission at 615 nm, sample window at 50 μs, time-per-flash at 100 μs, initial delay at 0.01 μs, number of flash at 10, and number of scan at 10, excitation collection from 700 to 900 nm.

### Example 5

### Fluorescence Decay of Up-Converting Fluorescence of the Probe Conjugates

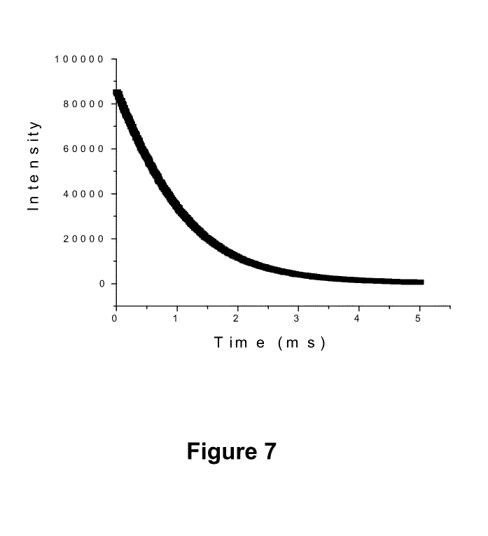

10 ng of the probe conjugates prepared in Example 2 is suspended in 600 μl water to in a cell. The decay is shown in FIG. 7. The following parameters are used to measure the fluorescence decay: excitation at 870 nm, emission at 615 nm, sample window at 50 μs, time-per-flash at 100 μs, initial delay at 0.01 μs, number of flash at 10, and number of scan at 10.

### Example 6

### Quantifying the Amount of Luteinizing Hormone (LH)

Each of six vials, designated as vial 1, 2, 3, 4, 5 and 6, respectively, contains the same amount of MP-β-Mab and a different amount of LH from Fitzgerald Industrial International, Inc., ranging from 0, 5, 20, 50, 100 and 200 ng in 500 μl of 50 mM PBS buffer (pH: 7.2) with 2 mg/ml BSA and 0.1% Tween 20. The samples are incubated for 20 minutes under gentle shaking. The vials are then placed in a magnetic device and almost all the magnetic particles are attracted to the vial walls close to the magnet. The supernatant is removed and the magnetic particles are re-suspended in 500 μl of 50 mM PBS buffer (pH: 7.2) with 2 mg/ml BSA and 0.1% Tween 20 after the vials were removed from the magnetic device. To each vial is added with a same amount of α-Mab-P and the mixtures are incubated for 20 minutes under gentle shaking. The magnetic particles are again separated from the rest of the mixtures by a magnetic device. The separated magnetic particles are washed three times by 500 μl of 50 mM PBS buffer (pH: 7.2) with 2 mg/ml BSA and 0.1% Tween 20. The washed magnetic particles in each vial are re-suspended in 500 μl of 50 mM PBS buffer (pH: 7.2) with 2 mg/ml BSA and 0.1% Tween 20 for time-resolved up-converting luminescence measurements. The time-resolved up-converting luminescence at 615 nm of each sample is measured at 20 μs delay by exciting the sample using 870 nm pulsed illumination. The relative intensity of the delayed up-converting luminescence at 615 nm is 150, 230, 407, 859, 1717, and 3553, for sample 1, 2, 3, 4, 5, and 6, respectively.

What is claimed:

1. A assay method for detecting the presence or quantity of an analyte in a sample, the assay method comprising:
a. contacting a sample containing the analyte with magnetic particles conjugated with a first specific binding member and a detection probe tagged with a second specific binding member, wherein the detection probe emitting luminescence at a shorter wavelength than the wavelength of an excitation light with a luminescence lifetime of more than 5 μsec, wherein the first specific binding member and the second specific binding member bind different epitopes of the analyte to form a sandwich complex;
b. separate the magnetic particles from the rest of the sample using a magnet device, wherein the magnetic particles include the sandwich complex;
c. exciting the separated magnetic particles using pulsed illumination at a first wavelength to obtain a detection signal by collecting and measuring the luminescence at a second wavelength after a certain period of time has elapsed following each pulse, wherein the second wavelength is shorter than the first wavelength.
d. comparing the detection signal with a calibration curve to obtain the amount of analyte in the sample, wherein the detection signal is proportional to the detection signal.

2. The method of claim 1, wherein the analyte is selected from a group consisting of proteins, peptides, microorganisms such as bacteria, viruses, yeasts, DNAs and RNAs, enzymes, antibodies and antigens

3. The method of claim 1, wherein the magnetic particles have sizes of from 10 nm to 10 μm which are either covalently or physically conjugated with the first binding member

4. The method of claim 1, wherein the first and second binding members include antibodies, antigens, DNAs and RNAs

5. The method of claim 1, wherein the detection probe include the chelates of lanthanide, particles encapsulated with the chelates of lanthanides, and the lanthanide-doped phosphor nanocrystal particles, wherein the lanthanide is selected from a group consisting of samarium, dysprosium, europium, terbium, and the combination of thereof.

6. The method of claim 1, wherein the detection probe absorbing photons of long wavelength and emitting one photon of a shorter wavelength with a emission lifetime of from about 20 μsec to 2000 μsec

7. The method of claim 1, wherein the pulsed illumination is generated through light emitting diodes, lasers, or tungsten lamps

8. The method of claim 1, where the luminescence was measured by a silicon photodiode and photomultiplier tube

9. The method of claim 1, wherein the pulsed illumination and the time-gated detection are controlled by timing circuitries

10. The method of claim 1, wherein the signal was measured after about 20 to 200 μsec of each pulse

11. A assay method for detecting the presence or quantity of an analyte in a sample, the assay method comprising:
a. contacting a sample containing the analyte with magnetic particles conjugated with a first specific binding member and a known amount of detection probe tagged with analyte or analyte analog wherein the detection probe is capable of emitting luminescence at a shorter wavelength than the wavelength of an excitation light with a luminescence lifetime of more than 5 μsec, wherein the first specific binding member bind specifically the analyte or analyte analog to form a complex;
b. separate the magnetic particles from the rest of the sample using a magnet device, wherein the magnetic particles include the sandwich complex;
c. exciting the separated magnetic particles using pulsed illumination at a first wavelength to obtain a detection signal by collecting and measuring the luminescence at a second wavelength after a certain period of time has elapsed following each pulse wherein the second wavelength is shorter than the first wavelength.
d. comparing the detection signal with a calibration curve to obtain the amount of analyte in the sample wherein the detection signal is reversely proportional to the detection signal.

12. The method of claim 11, wherein the analyte is selected from a group consisting of small molecules, proteins, peptides, microorganisms such as bacteria, viruses, yeasts, haptens, enzymes, antibodies and antigens

13. The method of claim 11, wherein the magnetic particles have sizes of from 10 nm to 10 μm which are either covalently or physically conjugated with the first binding member

14. The method of claim 11, wherein the first binding member is antibodies, antigens, DNAs or RNAs

15. The method of claim 11, wherein the detection probe is selected from a group consisting of the chelates of lanthanide, particles encapsulated with the chelates of lanthanides, and the lanthanide-doped phosphor nanocrystal particles, wherein the lanthanide is samarium, dysprosium, europium, terbium, or the combination of thereof.

16. The method of claim 11, wherein the detection probe absorbing two photons of long wavelength and emitting one photon of a shorter wavelength with a emission lifetime of from about 20 μsec to 2000 μsec

17. The method of claim 11, wherein the pulsed illumination is generated through light emitting diodes, lasers, or tungsten lamps

18. The method of claim 11, wherein the luminescence was measured by a silicon photodiode and photomultiplier tube

19. The method of claim 11, wherein the pulsed illumination and the time-gated detection are controlled by timing circuitries

20. The method of claim 11, wherein the signal was measured after about 20 to 200 μs of each pulse.
